(19) United States
(12) Patent Application Publication
(10) Pub. No.: US 2012/0308620 A1
(43) Pub. Date: Dec. 6, 2012

(54) TOPICAL COSMETIC PREPARATION CONTAINING ELASTOGENESIS INDUCING SUBSTANCES ASSOCIATED WITH A SYSTEMIC ABSORPTION RETARDANT

(75) Inventors: Filiberto P. Zadini, camarillo, CA (US); Giorgio C. Zadini, Camarillo, CA (US)

(73) Assignee: AtheroNova Operations, Inc., Irvine, CA (US)

(21) Appl. No.: 13/491,411

(22) Filed: Jun. 7, 2012

Related U.S. Application Data
(63) Continuation-in-part of application No. 12/592,032, filed on Nov. 18, 2009, now abandoned.

(51) Int. Cl. A61K 8/97; A61K 8/14; A61K 8/35; A61Q 19/08

(52) U.S. Cl. 424/401; 424/195.18; 424/725; 514/691

(57) ABSTRACT

A dermatological topical formulation such as a cream, ointment, or lotion containing an elastogenesis inducer and a systemic absorption retardant is described. Such a formulation achieves high local tissue concentration of the elastogenesis inducer in the vicinity of the application site, minimizes systemic absorption of the elastogenesis inducer, and maximizes a concentration of the elastogenesis inducer in dermis, resulting in formation or restoration of elastin fibers in adult skin. The formulation can further contain a skin penetration enhancer compound.

### RELATED APPLICATIONS

This application is a continuation-in-part of U.S. application Ser. No. 12/592,032, filed Nov. 18, 2009, which claims priority to U.S. Provisional Application No. 61/199,348, filed Nov. 18, 2008. The contents of each of the foregoing applications are hereby incorporated by reference herein in their entireties.

### FIELD

This application relates to cosmetic or dermatological topical formulations containing an elastogenesis inducer and a systemic absorption retardant.

### BACKGROUND

Elastin fibers are at least in part responsible for the elasticity of the skin. The process of aging of the skin is associated in part with gradual loss of elasticity resulting from deficiency of elastic fibers replacement in adult life. A number of enzymes of the lysyl oxidase family play an important role in the formation of elastic fibers. In particular, the enzymes lysyl oxidase (LOX) and lysyl oxidase-like (LOXL) are responsible for elastin cross-linking. It has been shown recently that LOXL is essential for the elastic fibers homeostasis and for their maintenance at adult age. It has been shown that LOXL plays an important role not only in elastic fibers formation but also in their renewal. LOXL appears to be associated with forming elastic fibers in the human skin. See Cenizo et al., Exp Dermatol 2006:15:574-581. It has also been shown that enzymes lysyl oxidase (LOX) and lysyl oxidase-like (LOXL), in particular LOXL, can be considered as a new target to re-induce elastogenesis. Specific substances, which include a few phytochemicals, have shown to be capable of re-inducing elastogenesis. Such substances are dill, currant, cardamon, black radish, small holly, cinnamon, lactic bacteria-based fermentations, oats, potato, silk, Asea foetida gum, ethyl hexenoate and its derivatives, methyl butyrate and its derivatives, and ethyl decadienoate and its derivatives.

In normal situations, however, once applied to the intact adult skin and transported into the dermis and dermal layer in their dermatological formulation after crossing the epidermal barrier, the elastogenesis inducers are promptly absorbed from the dermal layer into the systemic circulation. This rapid systemic absorption into the systemic circulation from the dermal layer, significantly limits the exposure of the of the LOX and LOXL to the elastogenesis inducers, which results in thwarting the overall effectiveness of these compounds.

### SUMMARY

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 2. The other clauses can be presented in a similar manner.

1. A topical dermatological formulation having an effective amount of at least one elastogenesis inducer and an effective amount of at least one systemic absorption retardant. In a related embodiment, the at least one systemic absorption retardant delays the absorption of said at least one elastogenesis inducer into the systemic circulation so as to maximize intradermal accumulation of said at least one elastogenesis-inducer substance.

2. The topical dermatological formulation of clause 1, wherein the at least one elastogenesis inducer comprises a phytochemical capable of inducing elastogenesis.

3. The topical dermatological formulation of clause 2, wherein the at least one elastogenesis inducer comprises a dill extract.

4. The topical dermatological formulation of clause 3, wherein the dill extract is at a concentration of 2% by weight based on the total weight of the formulation.

5. The topical dermatological formulation of clause 3, wherein the dill extract is at a concentration greater than 2% by weight based on the total weight of the formulation.

6. The topical dermatological formulation of clause 1, wherein the at least one elastogenesis inducer comprises ethoxyhexyl-bicyclooctanone (Ethocyn®).

7. The topical dermatological formulation of clause 1, wherein the at least one systemic absorption retardant comprises a mixture of at least two of benzyl alcohol, acetone or isopropanol.

8. The topical dermatological formulation of clause 7, wherein the mixture is also capable of inducing skin permeation enhancement.

9. The topical dermatological formulation of clause 1, wherein the formulation further comprises a percutaneous permeability enhancer.

10. The topical dermatological formulation of clause 9, wherein said percutaneous permeability enhancer comprises a chemical permeability enhancer.

11. The topical dermatological formulation of clause 10, wherein said percutaneous chemical permeability enhancer comprises a liposome.

12. The topical dermatological formulation of clause 10, wherein said percutaneous chemical permeability enhancer comprises a cyclodextrin.

13. The topical dermatological formulation of clause 10, wherein said percutaneous chemical permeability enhancer comprises a chemically induced heat.

14. The topical dermatological formulation of clause 9, wherein said percutaneous permeability enhancer comprises a physical permeability enhancer.

15. The topical dermatological formulation of clause 14, wherein said percutaneous physical permeability enhancer comprises a dermabrasion.

16. The topical dermatological formulation of clause 14, wherein said percutaneous physical permeability enhancer comprises a microdermabrasion.

17. The topical dermatological formulation of clause 14, wherein said percutaneous physical permeability enhancer comprises an iontophoresis.

18. The topical dermatological formulation of clause 14, wherein said percutaneous physical permeability enhancer comprises a microneedle.

19. The topical dermatological formulation of clause 14, wherein said percutaneous physical permeability enhancer comprises electroporation.

20. The topical dermatological formulation of clause 14, wherein said percutaneous physical permeability enhancer comprises a sonophoresis.

21. The topical dermatological formulation of clause 14, wherein said percutaneous physical permeability enhancer comprises a physically induced heat.

22. A method of treating conditions associated with loss of elastic fibers in the skin of a subject in need thereof, said method comprises administering to the subject a topical formulation having a therapeutically effective amount of at least one elastogenesis inducer and a therapeutically effective amount of at least one systemic absorption retardant. In a related embodiment, the at least one systemic absorption retardant delays the absorption of said at least one elastogenesis inducer into the systemic circulation so as to maximize intradermal accumulation of said at least one elastogenesis-inducer substance.

23. The method of clause 22, wherein the at least one elastogenesis inducer comprises a phytochemical capable of inducing elastogenesis.

24. The method of clause 23, wherein the at least one elastogenesis inducer comprises a dill extract.

25. The method of clause 24, wherein the dill extract is at a concentration of 2% by weight based on the total weight of the formulation.

26. The method of clause 24, wherein the dill extract is at a concentration greater than 2% by weight based on the total weight of the formulation.

27. The method of clause 22, wherein the at least one elastogenesis inducer comprises ethoxyhexyl-bicyclooctanone (Ethocyn®).

28. The method of clause 22, wherein the at least one systemic absorption retardant comprises a mixture of at least two of benzyl alcohol, acetone or isopropanol.

29. The method of clause 28, wherein the mixture is capable of inducing permeation enhancement.

30. The method of clause 22, wherein the formulation further comprises a percutaneous permeability enhancer.

31. The method of clause 30, wherein said percutaneous permeability enhancer comprises a chemical permeability enhancer.

32. The method of clause 31, wherein said percutaneous chemical permeability enhancer comprises a liposome.

33. The method of clause 31, wherein said percutaneous chemical permeability enhancer comprises a cyclodextrin.

34. The method of clause 31, wherein said percutaneous chemical permeability enhancer comprises a chemically induced heat.

35. The method of clause 30, wherein said percutaneous permeability enhancer comprises a physical permeability enhancer.

36. The method of clause 35, wherein said percutaneous physical permeability enhancer comprises a dermabrasion.

37. The method of clause 35, wherein said percutaneous physical permeability enhancer comprises a microdermabrasion.

38. The method of clause 35, wherein said percutaneous physical permeability enhancer is an iontophoresis.

39. The method of clause 35, wherein said percutaneous physical permeability enhancer comprises a microneedles apparatus.

40. The method of clause 35, wherein said percutaneous physical permeability enhancer comprises electroporation.

41. The method of clause 35, wherein said percutaneous physical permeability enhancer comprises a sonophoresis.

42. The method of clause 35, wherein said percutaneous physical permeability enhancer comprises a physically induced heat.

43. A topical dermatological formulation comprising:
(a) an effective amount of at least one elastogenesis inducer comprising at least one of dill extract, currant extract, cardamom extract, black radish extract, small holly extract, cinnamon extract, oats extract, potato extract, silk extract, asafoetida gum, ethyl hexenoate, derivatives of ethyl hexenoate, methyl butyrate, derivatives of methyl butryate, ethyl decadienoate, derivatives of decadienoate, ethoxyhexyl-bicyclooctanone (Ethocyn®), aloe vera, or glycolic acid; and
(b) an effective amount of at least one systemic absorption retardant comprising at least two of benzyl alcohol, acetone or isopropanol.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 shows a liposome with a payload containing elastogenesis inducing substances associated with an absorption retardant.

FIG. 2 shows a cyclodextrin with a payload containing elastogenesis inducing substances associated with an absorption retardant.

### DETAILED DESCRIPTION

In the following detailed description, numerous specific details are set forth to provide a full understanding of the subject technology. It will be apparent, however, to one ordinarily skilled in the art that the subject technology may be practiced without some of these specific details. In other instances, well-known structures and techniques have not been shown in detail so as not to obscure the subject technology.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa.

In describing and claiming the present invention, the following terminology will be used in accordance with the definitions set out below.

“Carriers” or “vehicles” as used herein refer to carrier materials suitable for transdermal or topical drug administration. Carriers and vehicles useful herein include any such materials known in the art, which are nontoxic and do not interact with other components of the composition in a deleterious manner.

“Effective amount” or “a therapeutically effective amount” of a therapeutically active agent is intended to mean a nontoxic but sufficient amount of a therapeutically active agent to provide the desired therapeutic effect. The amount that is effective will vary from subject to subject, depending on the age and general condition of the individual, the particular active agent or agents, and the like. Thus, it is not always possible to specify an exact effective amount. However, an appropriate effective amount in any individual case may be determined by one of ordinary skill in the art using routine experimentation. Furthermore, the exact effective amount of an active agent incorporated into a composition or dosage form of the invention is not critical, so long as the concentration is within a range sufficient to permit ready application of the formulation so as to deliver an amount of the active agent that is within a therapeutically effective range. The terms “effective amount” or “therapeutically effective amount” of an elastogenesis inducer, refer to an amount that results in a detectable and statistically significant net increase in elastin fiber formation according to accepted criteria and methodologies that may vary according to the specific application of the method, for example, by northern blot analysis for increased transcription of tropoelastin-encoding mRNA, by immunohistochemical detection of increased elastin fibers in a sample from a cultured cell or from a subject, by western immunoblot analysis for one or more elastin fiber protein components in a sample from a culture cell or from a subject, or by other criteria as will be known to those familiar with the relevant art. The terms “effective amount” or “therapeutically effective amount” of a systemic absorption retardant, refer to an amount of a systemic absorption retardant that upon administration achieves a desired result, e.g., slowing, inhibiting or preventing the systemic absorption of elastogenesis inducer(s) through dermal microvasculature, and/or allowing the concentration of elastognesis inducer(s) to maximize in the dermis, intradermal or dermal layer.

As used herein, the term “maximize” in relation to interadermal accumulation of elastogenesis inducer(s), relates to an increase in the interadermal concentration of elastogenesis inducer(s), i.e., an increase in the rate or magnitude at which the elastogenesis inducer(s), in the presence of a systemic absorption retardant, accumulates in a predetermined volume of dermis below the area of topical administration relative to the rate or magnitude that would be obtained in the absence of the systemic absorption retardant. The enhanced accumulation effected through the use of such systemic absorption retardants can be observed by, for example, measuring the effective amount of the elastogenesis inducer(s) in presence or absence of the systemic absorption retardant.

The term “elastogenesis inducer,” as used herein, refers to any agent that induces, enhances, promotes or increases a specific activity, such as elastogenesis or formation of elastin fibers in a subject. Typical elastogenesis inducers as used herein include dill extract, or extracts from currant, cardamon, black radish, small holly, cinnamon, lactic bacteria-based fermentations, oats, potato, silk and asafoetida gum. Other typical elastogenesis inducers as used herein include ethyl hexenoate and its derivatives, methyl butyrate and its derivatives, ethyl decadienoate and its derivatives, Ethocyn®, aloe vera, and glycolic acid. The term “induce” in this context should not be interpreted to only mean induction (i.e., to start from a stop or stagnant state); rather the term should be interpreted broadly to mean enhancement, promotion and/or increase as well.

As use herein, the term “systemic absorption retardant” refer to a substance or combination of substances that can slow, prevent or inhibit the absorption of elastogenesis inducer(s) from dermis or dermal layer or intradermal layer into the systemic blood circulation. For example, a mixture of benzyl alcohol, acetone and isopropanol is a systemic absorption retardant that slows or inhibits the systemic absorption of the elastogenesis inducer(s) from dermis into the systemic blood circulation.

“Effective amount” or “an effective permeation enhancing amount” of a permeation enhancer refers to a nontoxic, non-damaging but sufficient amount of the enhancer composition to provide the desired increase in skin permeability and, correspondingly, the desired depth of penetration, rate of administration, and amount of drug delivered.

“Penetration enhancement” or “permeation enhancement” as used herein relates to an increase in the permeability of the skin or mucosal tissue to the selected pharmacologically active agent, i.e., so that the rate at which the agent permeates therethrough (i.e., the “flux” of the agent through the body surface) is increased relative to the rate that would be obtained in the absence of permeation enhancer. The enhanced permeation effected through the use of such enhancers can be observed by measuring the rate of diffusion of drug through animal or human skin using, for example a Franz diffusion apparatus as known in the art.

“Predetermined area” of skin or mucosal tissue refers to the area of skin or mucosal tissue through which a drug-enhancer formulation is delivered, and is a defined area of intact unbroken living skin or mucosal tissue. That area will usually be in the range of about 5-200 cm2, more usually in the range of about 5-100 cm2, preferably in the range of about 20-60 cm2. However, it will be appreciated by those skilled in the art of drug delivery that the area of skin or mucosal tissue through which drug is administered may vary significantly, depending on patch configuration, dose, and the like.

“Topical administration” is used in its conventional sense to mean delivery of a topical drug or pharmacologically active agent to the skin or mucosa, as in, for example, the treatment of various skin disorders.

“Treating” and “treatment” as used herein refer to reduction in severity and/or frequency of symptoms, elimination of symptoms and/or underlying cause, prevention of the occurrence of symptoms and/or their underlying cause, and improvement or remediation of damage. The present method of “treating” a patient, as the term is used herein, thus encompasses both prevention of a disorder in a predisposed individual and treatment of the disorder in a clinically symptomatic individual.

The major component of elastic fibers is an amorphous polymer composed of the polypeptide elastin (known as tropoelastin when in monomeric form). Polymerization of elastic fibers requires an initial step of oxidative deamination of lysine residues, which is catalyzed by a lysyl oxidase. The resulting aldehyde groups condense spontaneously with adjacent aldehydes or ε-amino groups of peptidyl lysine to form covalent cross-linkages. Lysyl oxidases are copper-dependent monoamine oxidases secreted by fibrogenic cells including fibroblasts and smooth muscle cells. Mammalian genomes have up to five potential LOX family members coding for the prototypic LOX and LOX-like polypeptides 1 through 4 (LOXL1-4). It has been shown that lysyl oxidases play an important role in elastic fiber homeostasis in adult tissues and that compounds that increase the activity of these enzymes are beneficial for inducing elastogenesis and treating or preventing conditions associated with a loss of elastic fibers.

Specific substances, which include a few phytochemicals, have shown the capability of re-inducing elastogenesis in adult skin. Such substances include dill, currant, cardamon, black radish, small holly, cinnamon, lactic bacteria-based fermentations, oats, potato, silk, asafoetida gum, ethyl hexenoate and its derivatives, methyl butyrate and its derivatives, and ethyl decadienoate and its derivatives. In particular, a dill extract has been shown to be the most effective in re-inducing elastogenesis in adult skin when applied topically. Another elastogenesis inducer substance is Ethocyn®, its chemical name being ethoxyhexyl-bicyclooctanone. Ethocyn® is claimed to stimulate the synthesis of elastin in the skin.

As discussed in the background section; however, once applied to the intact adult skin and transported into the dermis and dermal layer in their dermatological formulation after crossing the epidermal barrier, the elastogenesis inducers are promptly absorbed from the dermal layer into the systemic circulation.

Accordingly, there remains a need for new topical dermatological or cosmetic formulations that can achieve high dermal or local tissue concentration of the elastogenesis inducers with minimum systemic absorption into the blood circulation, over a period of time sufficient to induce elastogenesis.

Thus, an object of the subject technology is to provide a cosmetic formulation capable of enhancing availability of the ingredients capable of re-induce elastogenesis in the skin, ultimately improving the skin texture and appearance.

Another object of the subject technology is to provide a cosmetic formulation with a mixture of ingredients capable of favoring local accumulation of the active cosmetic ingredients with maximization of the local effect of the active ingredients.

Another object of the subject technology is to provide a cosmetic formulation with a mixture of ingredients capable of favoring local accumulation of the active cosmetic ingredients via a decrease of clearance of such active cosmetic ingredients from the dermal layer and via enhancement of percutaneous transdermal delivery into the dermal layer to maximize accumulation of active cosmetic ingredients in the dermal layer.

In an embodiment, the subject technology provides a topical or cosmetic formulation containing at least one elastogenesis inducer and at least one systemic absorption retardant to achieve a high local tissue concentration of the elastogenesis inducer in dermal layer. In a related embodiment, the at least one systemic absorption retardant compound is in an effective amount to slow, prevent or inhibit systemic absorption of the at least one elastogenesis inducer of the formulation. In another related embodiment, the at least one elastogenesis inducer is in an effective amount to induce elastogenesis in dermis and dermal layer of skin. In another related embodiment, the at least one elastogenesis inducer includes compounds such as dill extract. In another related embodiment, the at least one systemic absorption retardant includes compounds such as a mixture of benzyl alcohol, acetone and isopropanol. In another related embodiment, the formulation contains at least two of benzyl alcohol, acetone or isopropanol as systemic absorption retardants.

Accordingly to the subject technology, the purpose of combining at least one elastogenesis inducer and at least one systemic absorption retardant is to maximize retention of the elastogenesis inducer locally in the dermis and minimize its systemic absorption from the dermal layer. Further addition of skin permeability enhancers to the formulations of the subject technology results in permeation enhancement of the elastogenesis inducer(s) in the dermis or dermal layer. Compounds such as benzyl alcohol, acetone or isopropanol and/or a mixture thereof can maximize both delivery and retention of elastogenesis inducer(s) of the subject technology. An increased intradermal concentration or accumulation of the elastogenesis inducer(s) can achieve an enhanced elastogenesis and/or enhanced restoration of the elastic fibers in the skin.

Indeed, in the absence of systemic absorption retardants, the elastogenesis inducers do not remain in the dermis and subcutaneous layers long enough in sufficient concentration to effectively induce elastogenesis in the skin. Without systemic absorption retardants, the elastogenesis inducers can hardly accumulate in the dermis to function as depot drug delivery system. The addition of a systemic or dermal layer absorption retardant, which is capable of hindering the systemic absorption of the elastogenesis inducers, can result in accumulation of the inducers in the dermal layer. A high local tissue concentration of the elastogenesis inducers in dermal layer maximizes the topical elastogenesis effect of these inducers. This accumulation of the elastogenesis inducers in the dermal layer further prolongs the exposure of the LOX and LOXL to such inducers so as to achieve an effective elastogenesis induction.

In an embodiment, the pharmaceutical formulations of the subject technology further includes at least one percutaneous delivery enhancer to facilitate the penetration of the elastogenesis inducers and/or the systemic absorption retardant into the dermis or dermal layer through the skin barrier represented by the epidermal stratum corneum. In a related embodiment, the at least one percutaneous delivery enhancer includes compounds such as cyclodextrins, liposomes, ethosomes.

In an embodiment, the subject technology relates to a topical cosmetic or dermatological formulation such as a cream or ointment or lotion or the likes, containing an effective amount of at least one elastogenesis inducer and an effective amount of at least one systemic absorption retardant. In a related embodiment, the topical formulation contains at least one systemic absorption retardant in an amount effective to retard or minimize the systemic absorption or clearance of the elastogenesis inducer(s) of the formulation from the dermal layer allowing the inducer(s) to remain longer and/or at a greater local concentration in dermis or dermal layer of skin. In another embodiment, the subject technology relates to the use of percutaneous delivery enhancers that can be added to the elastogenesis inducer-systemic absorption retardant combinations so as to enhance passage of these compounds into the dermis or dermal layer.

In an embodiment, the subject technology relates to a topical formulation such as cosmetic cream containing a mixture of three components—benzyl alcohol, acetone and isopropanol, as a systemic absorption retardant—and an elastogenesis inducer. The benzyl alcohol, acetone and isopropanol mixture represents an ideal addition to the cosmetic formulations of the subject technology for rebuilding the elastic fibers network in the skin because it not only acts as a systemic absorption retardant delaying the clearance of the elastogenesis inducers from the dermal layer, but also acts as a percutaneous transdermal delivery enhancer, promoting conveyance of the elastogenesis inducer into the dermis and dermal layer with an overall effect of accumulation of elastogenesis inducer precisely where it is required to be, with a resulting maximization of the elastogenesis effect. In an embodiment, the formulation contains at least two of benzyl alcohol, acetone or isopropanol as systemic absorption retardants.

The effectiveness of the mixture of benzyl alcohol, acetone and isopropanol as a systemic absorption retardant has been shown in connection with erythromycin. See Peng et al., “delivery of erythromycin to dermal layers in rats by means of a transphase delivery system,” J Pharm Pharmacol. 1999 October; 51(10):1135-41. It was shown in Peng et al. that the mixture of benzyl alcohol, acetone and isopropanol worked as both a systemic absorption retardant and a transdermal permeability enhancer.

Without being bound by theory, the mechanism by which the topical formulations of the subject technology work involves combining an elastogenesis inducer with a systemic absorption retardant agent which can hinder, slow, prevent or inhibit the systemic absorption of elastogenesis inducer of the formulations from the dermal tissues. The delay in or lack of systemic absorption of the elastogenesis inducers will result in significant and persistent local accumulation of these compounds in the dermis and dermal layer as the elastogenesis inducers are transdermally delivered across the stratum corneum of the epidermis after topical administration of the formulations. The systemic absorption retardants of the subject formulations will facilitate an increase in local concentration of the elastogenesis inducers in dermis. Due to lack of or limited absorption of the elastogenesis inducers into the systemic circulation, high local concentration of the elastogenesis inducers will be sustained over a period of time, which will in turn maximizes the local effect of the elastogenesis inducers in inducing elastogenesis and treating or preventing conditions associated with a loss of elastic fibers.

In an embodiment, the formulations of the subject technology achieve a sustained dermis levels of elastogenesis inducer(s) for a period of at least two hours. Alternatively or in addition, the sustained dermis levels of elastogenesis inducer(s) is maintained at a therapeutically effective amount for at least two hours. In another embodiment, the formulations of the subject technology achieve a sustained dermis levels of elastogenesis inducer(s) for a period of at least four hours. Alternatively or in addition, the sustained dermis levels of elastogenesis inducer(s) is maintained at a therapeutically effective amount for at least four hours. As described in above, the therapeutically effective amount of an elastogenesis inducer in the dermal layer can be measured by methods generally known in the art.

In an embodiment, the subject technology provides a topical pharmaceutical formation such as a cream or ointment or lotion or the like for treating conditions associated with a loss of elastic fibers, containing an effective amount of at least one elastogenesis inducer and an effective amount of at least one systemic absorption retardant. In a related embodiment, the topical formulation contains at least one systemic absorption retardant in an amount effective to retard the systemic absorption or clearance of the elastogenesis inducer(s) of the formulation from the dermal layer allowing the inducer(s) to remain longer and/or at a greater local concentration in the dermis or dermal layer of the skin. In another embodiment, the subject technology relates to the use of percutaneous delivery enhancers that can be added to the elastogenesis inducer-systemic absorption retardant combinations so as to enhance passage of elastogenesis inducer(s) into the dermis and dermal layer.

The formulations of the subject technology further contain or will be administered together with a transdermal delivery enhancer. In a related embodiment, the transdermal delivery enhancer can be a chemical entity such as a compound of genera cyclodextrins, liposomes, ethosomes, sulfoxides and the like. In another related embodiment, the transdermal delivery enhancement of the formulations of the subject technology can be achieved by a physical means such as iontophoresis, electroporation, sonophoresis, thermal poration and the like.

In an embodiment, the subject technology further provides a method for restoring elasticity of adult skin by topically administering to a subject in need thereof a formulation such as a cream or ointment or lotion or the likes, containing an effective amount of at least one elastogenesis inducer and an effective amount of at least one systemic absorption retardant. In a related embodiment, the topical formulation contains at least one systemic absorption retardant in an amount effective to retard the systemic absorption of the elastogenesis inducer(s) of the formulation from the dermal layer allowing the inducer(s) to remain longer and/or at a greater local concentration in dermis or dermal layer of skin. In another embodiment, the subject technology relates to the use of percutaneous delivery enhancers that can be added to the elastogenesis inducer-systemic absorption retardant combination so as to enhance passage of elastogenesis inducer(s) into the dermis and dermal layer.

In an embodiment, the subject technology further provides a method treating conditions associated with a loss of elastic fibers in skin, said method includes topically administering to a subject in need thereof a formulation such as a cream or ointment or lotion or the likes, containing an effective amount of at least one elastogenesis inducer and an effective amount of at least one systemic absorption retardant. In a related embodiment, the topical formulation contains at least one systemic absorption retardant in an amount effective to retard the systemic absorption of the elastogenesis inducer(s) of the formulation from the dermal layer allowing the inducer(s) to remain longer and/or at a greater local concentration in dermis or dermal layer of skin. In another embodiment, the subject technology relates to the use of percutaneous delivery enhancers that can be added to the elastogenesis inducer-systemic absorption retardant combination so as to enhance passage of elastogenesis inducer(s) into the dermis and dermal layer.

Elastogenesis Inducers

The elastogenesis inducers that can be used in the formulations of the subject technology include, but not limited to dill extract, or other compounds having the same characteristic of inducing elastogenesis in the skin such as currant, cardamon, black radish, small holly, cinnamon, lactic bacteria-based fermentations, oats, potato, silk, Asea foetida gum, ethyl hexenoate and its derivatives, methyl butyrate and its derivatives, ethyl decadienoate and its derivatives, said elastogenesis inducing ingredients being associated with a local systemic absorption retardant or retardants. Other compounds or compositions that can induce elastogenesis and may be used in the topical formulations of the subject technology are those disclosed in U.S. Pat. No. 7,666,829, and U.S. Patent Publication No. 20100197563, the contents of which are incorporated herein by reference.

Another elastogenesis inducer to be used with or in association with a systemic absorption retardant is Ethocyn®, chemical name ethoxyhexyl-bicyclooctanone. As pointed out above, Ethocyn® is claimed to stimulate the synthesis of elastin in the skin. As per its mechanism of action, Ethocyn® is claimed to block the action of the androgenic sex hormone DHT, i.e. dehydrotestosterone. It is a well-established fact that DHT levels increase with age. Ethocyn® makers claim that “age-related decline of elastin synthesis is caused mainly by high levels of DHT. Therefore, topical application of Ethocyn® presumably blocks DHT in the skin and thereby stimulates elastin synthesis.”

The elastogenesis inducer(s) employed in the formulations of the subject technology are present in a therapeutically effective concentration, for example, at least 0.01% by weight based on the total weight of the formulation. In general, the elastogenesis inducer(s) will be present in an amount of equal to or greater than 0.05%, or in an amount equal to or greater than 2%, or in an amount equal to or greater than 5%, or in an amount equal to or greater than 10%, or in an amount equal to or greater than 15%, or in an amount equal to or greater than 25%, or in an amount equal to or greater than 50%, by weight based on the total weight of the formulation.

As described above, the formulations of the subject technology contain elastogenesis inducer(s) in a therapeutically effective concentration or amount which results in a detectable and statistically significant increase in elastin fiber formation according to accepted criteria and methodologies that may vary according to the specific application of the method, for example, by northern blot analysis for increased transcription of tropoelastin-encoding mRNA, by immunohistochemical detection of increased elastin fibers in a sample from a cultured cell or from a subject, by western immunoblot analysis for one or more elastin fiber protein components in a sample from a culture cell or from a subject, or by other criteria as will be known to those familiar with the relevant art. It will be evident to those skilled in the art that the number and frequency of administrations will be dependent upon the response of a subject to treatment with the formulations of the subject technology (or upon the resulting evidence of induced elastogenesis in the subject), from which may be determined what is an effective amount.

Systemic Absorption Retardant

The systemic absorption retardants that can be used in the formulations of the subject technology include, but not limited to a mixture of benzyl alcohol, acetone and isopropanol, or other compounds having the same characteristic of minimizing systemic absorption of elastogenesis inducer(s) from dermis or dermal layer. The mixture of benzyl alcohol, acetone and isopropanol when combined with the elastogenesis inducer will promote transport of the elastogenesis inducer across the epidermal barrier while at the same time such a mixture will minimize systemic absorption of the inducer, which results in significant and persistent accumulation of the elastogenesis inducer in the dermis and in the subcutaneous tissue. Such a formulation, according to an embodiment of the subject technology, induces a high local tissue concentration of the elastogenesis inducer in the vicinity of the site of application, and, consequently, can maximize the local pharmacological effects of the elastogenesis inducing substances in the dermis and dermal layer in the proximity of the site of application, and ultimately result in recreating or restoring skin elasticity at the site of application.

The systemic absorption retardant(s) employed in the formulations of the subject technology are present in a therapeutically effective concentration, for example, at least 0.01% by weight based on the total weight of the formulation. In general, the therapeutic agent will be present in an amount of equal to or greater than 0.05%, or in an amount equal to or greater than 2%, or in an amount equal to or greater than 5%, or in an amount equal to or greater than 10%, or in an amount equal to or greater than 15%, or in an amount equal to or greater than 25%, or in an amount equal to or greater than 50%, by weight based on the total weight of the formulation. In an embodiment, the systemic absorption retardant is a mixture of benzyl alcohol (10%), acetone (40%), isopropanol (50%).

In an embodiment, the formulation of the subject technology contains at least two of benzyl alcohol, acetone or isopropanol as systemic absorption retardants. In another embodiment, the systemic absorption retardant of the subject technology acts also as a skin penetration enhancer.

Skin Permeability Enhancer

The skin permeability enhancer or percutaneous permeability enhancer that can be used in the formulations of the subject technology result in permeation or penetration enhancement of the elastogenesis inducer(s) and systemic absorption retardant(s) and include, but not limited to percutaneous chemical enhancers and percutaneous physical enhancers.

The percutaneous chemical enhancers which can be added to the formulations of the subject technology can be classified as cyclodextrins, liposomes, ethosomes, sulfoxides, alcohols, fatty acids, fatty acid esters, polyols, amides surfactants, terpene, alkanones and organic acids.

In particular, the percutaneous chemical enhancers that are suitable for addition to the formulations of the subject technology are ethanol, glyceryl monoethyl ether, monoglycerides, isopropylmyristate, lauryl alcohol (also, lauric acid, lauryl lactate), terpinol, menthol, D-limonene, beta-cyclodextrin, DMSO, polysorbates, fatty acids e.g. oleic, N-methylpyrrolidone, polyglycosylated glycerides, 1-dodecylaza cycloheptan-2-one known as Azone®, cyclopentadecalactone known as CPE-215®, alkyl-2-(N,N-disubstituted amino)-alkanoate ester known as NexAct®, and 2-(n-nonyl)-1,3-dioxolane known as SEPA®.

In an embodiment, the skin permeability enhancer is a mixture of at least two of benzyl alcohol, acetone or isopropanol.

In addition to chemical transdermal permeability enhancers, physical transdermal enhancers can be used. Physical enhancers which can be used are iontophoresis, electroporation, sonophoresis, thermal poration and in general physically/chemically induced heat, microneedles, dermabrasion. Topical delivery into the dermal layers of elastogenesis inducing substances can also be enhanced after microdermabrasion, microdermabrasion can be used alone or in combination with percutaneous chemical enhancers, such as the percutaneous chemical enhancers described above and/or with physical enhancers, such as the physical enhancers described above.

As described above, in an embodiment, the subject technology relates to a topical cosmetic or dermatological formulation such as a cream or ointment or lotion or the likes, containing an effective amount of at least one elastogenesis inducer and an effective amount of at least one systemic absorption retardant. In a related embodiment, the topical formulation contains at least one systemic absorption retardant in an amount effective to retard the systemic absorption of the elastogenesis inducer(s) of the formulation from the dermal layer allowing the inducer(s) to remain longer and/or at a greater local concentration in dermis or dermal layer of skin. In another embodiment, the subject technology relates to the use of percutaneous delivery enhancers that can be added to the elastogenesis inducer-systemic absorption retardant combinations so as to enhance passage of elastogenesis inducer(s) into the dermis and dermal layer.

Suitable formulations of the subject technology include ointments, creams, gels, lotions, solutions, pastes, and the like. Ointments, as is well known in the art of pharmaceutical formulation, are semisolid preparations that are typically based on petrolatum or other petroleum derivatives. The specific ointment foundation to be used, as will be appreciated by those skilled in the art, is one that will provide for optimum drug delivery, and, preferably, will provide for other desired characteristics as well, e.g., emolliency or the like. As with other carriers or vehicles, the ointment foundation should be inert, stable, nonirritating and nonsensitizing. As explained in Remington: The Science and Practice of Pharmacy, 20th edition (Lippincott Williams & Wilkins, 2000), ointment foundations may be grouped in four classes: oleaginous, emulsifiable, emulsion, and water-soluble. Oleaginous ointment foundations include, for example, vegetable oils, fats obtained from animals, and semisolid hydrocarbons obtained from petroleum. Emulsifiable ointment foundations, also known as absorbent ointment foundations, contain little or no water and include, for example, hydroxystearin sulfate, anhydrous lanolin and hydrophilic petrolatum. Emulsion ointment foundations are either water-in-oil (W/O) emulsions or oil-in-water (O/W) emulsions, and include, for example, cetyl alcohol, glyceryl monostearate, lanolin and stearic acid. Preferred water-soluble ointment foundations are prepared from polyethylene glycols of varying molecular weight.

Creams, as also well known in the art, are viscous liquids or semisolid emulsions, either oil-in-water or water-in-oil. Cream foundations are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions, which are preferred for delivery of cosmetic agents, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solute) in another liquid such that the molecules of the dissolved substance are dispersed among those of the solvent. The solution may contain other pharmaceutically acceptable chemicals to buffer, stabilize or preserve the solute. Commonly used examples of solvents used in preparing solutions are ethanol, water, propylene glycol or any other pharmaceutically acceptable vehicle.

Pastes are semisolid dosage forms in which the active agent is suspended in a suitable foundation. Depending on the nature of the foundation, pastes are divided between fatty pastes or those made from single-phase, aqueous gels. The foundation in a fatty paste is generally petrolatum or hydrophilic petrolatum or the like. The pastes made from single-phase aqueous gels generally incorporate carboxymethylcellulose or the like as the foundation.

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N-[1-2,3-dioleyloxy)propyl]-N,N,N-triethylammonium liposomes are available under the tradename Lipofectin® (GIBCO BRL, Grand Island, N.Y.). Anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline, dioleoylphosphatidyl glycerol, dioleoylphoshatidyl ethanolamine, among others. These materials can also be mixed with N-[1-2,3-dioleyloxy)propyl]-N,N,N-triethylammonium (DOTMA) in appropriate ratios. Methods for making liposomes using these materials are well known in the art.

Micelles are known in the art and are comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. They are generally, although not necessarily, formed from lipids, preferably charged lipids such as phospholipids. Preparation of lipidic microspheres is well known in the art and described in the pertinent texts and literature.

Various additives, known to those skilled in the art, may be included in the topical formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The formulation may also contain irritation-mitigating additives to minimize or eliminate the possibility of skin irritation or skin damage resulting from the drug, the base enhancer, or other components of the formulation. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the formulation at a concentration effective to mitigate irritation or skin damage, typically representing not more than about 20 wt %, more typically not more than about 5 wt %, of the formulation.

The concentration of the active agent in the formulation will typically depend upon a variety of factors, including the disease or condition to be treated, the nature and activity of the active agent, the desired effect, possible adverse reactions, the ability and speed of the active agent to reach its intended target, and other factors within the particular knowledge of the patient and physician. Preferred formulations will typically contain on the order of about 0.5-50 wt %, preferably about 5-30 wt %, active agent, i.e., an elastogenesis inducer.

Drug Delivery Systems

An alternative method for delivering the formulations of the subject technology involves the use of a drug delivery system, e.g., a topical or transdermal “patch,” wherein the formulation is contained within a laminated structure that is to be affixed to the skin. In such a structure, the formulation is contained in a layer, or “reservoir,” underlying an upper backing layer that serves as the outer surface of the device during use. The laminated structure may contain a single reservoir, or it may contain multiple reservoirs.

In one embodiment, the formulation reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the active agent, inorganic or organic base, and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing the drug, the base enhancer, or other components of the formulation contained within the device. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure preferably includes a release liner. Immediately prior to use, this layer is removed from the device so that the system may be affixed to the skin. The release liner should be made from a drug/vehicle impermeable material, and is a disposable element, which serves only to protect the device prior to application. Typically, the release liner is formed from a material impermeable to the pharmacologically active agent and the base enhancer, and is easily stripped from the transdermal patch prior to use.

In an alternative embodiment, the drug-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or pouch, or it may be a hydrogel reservoir, or may take some other form. Hydrogel reservoirs are particularly preferred herein. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be a drug, a base enhancer, an additional enhancer, or some other component contained in the drug delivery system.

A rate-controlling membrane, if present, will be included in the system on the skin side of one or more of the drug reservoirs. The material used to form such a membrane is selected so as to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 5-200 cm2, preferably 5-100 cm2, more preferably 20-60 cm2. That area will vary, of course, with the amount of drug to be delivered and the flux of the drug through the body surface. Larger patches can be used to accommodate larger quantities of drug, while smaller patches can be used for smaller quantities of drug and/or drugs that exhibit a relatively high permeation rate.

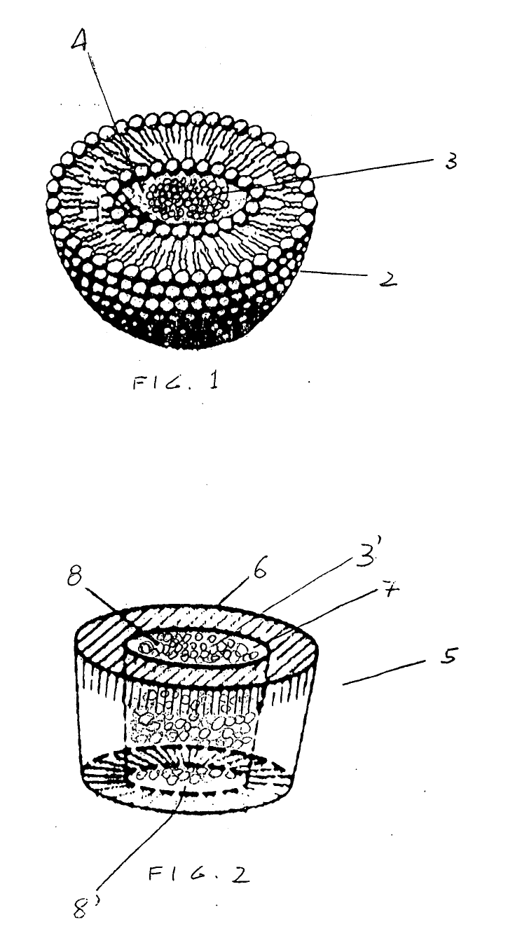

By way of illustration and not limitation, in some embodiments, as shown in FIG. 1, liposome vesicle 1 is of spherical shape with multilayer phospholipids membrane wall 2 delimiting inner cavity 4 for payload 3. Payload 3 can be any of the above mentioned elastogenesis enhancers such as dill extract or currant, cardamon, black radish, small holly, cinnamon, lactic bacteria-based fermentations, oats, potato, silk, asafoetida gum, ethyl hexenoate and its derivatives, methyl butyrate and its derivatives, ethyl decadienoate and its derivatives, alone or in a mixture or Ethocyn® said elastogenesis inducing ingredients being associated within the liposome vesicle with a benzyl alcohol, acetone and isopropanol mixture in the concentration disclosed above.

By way of illustration and not limitation, in some embodiments, FIG. 2, for example, shows cyclodextrin unit 5 generally of toroid shape having polysaccharide wall 6 delimiting cavity 7 for payload 3′. Toroid shaped cyclodextrin 5 is formed with upper larger opening 8 and lower smaller opening 8A. Payload 3′ housed within cavity 7, is the same as for liposome 1.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. A topical dermatological formulation comprising:
(a) an effective amount of at least one elastogenesis inducer comprising at least one of dill extract, currant extract, cardamom extract, black radish extract, small holly extract, cinnamon extract, oats extract, potato extract, silk extract, asafoetida gum, ethyl hexenoate, derivatives of ethyl hexenoate, methyl butyrate, derivatives of methyl butryate, ethyl decadienoate, derivatives of decadienoate, ethoxyhexyl-bicyclooctanone; and
(b) an effective amount of at least one systemic absorption retardant comprising at least two of benzyl alcohol, acetone, or isopropanol.

2. The topical dermatological formulation of claim 1, wherein the at least one elastogenesis inducer comprises a dill extract.

3. The topical dermatological formulation of claim 2, wherein the dill extract is at a concentration of 2% by weight based on the total weight of the formulation.

4. The topical dermatological formulation of claim 2, wherein the dill extract is at a concentration greater than 2% by weight based on the total weight of the formulation.

5. The topical dermatological formulation of claim 1, wherein the at least one elastogenesis inducer comprises ethoxyhexyl-bicyclooctanone (Ethocyn®).

6. The topical dermatological formulation of claim 1, wherein the at least one systemic absorption retardant comprises a mixture of benzyl alcohol, acetone or isopropanol.

7. The topical dermatological formulation of claim 6, wherein the mixture is also capable of inducing skin permeation enhancement.

8. The topical dermatological formulation of claim 1, wherein the formulation further comprises a percutaneous permeability enhancer.

9. The topical dermatological formulation of claim 8, wherein said percutaneous permeability enhancer comprises a chemical permeability enhancer.

10. The topical dermatological formulation of claim 9, wherein said percutaneous chemical permeability enhancer comprises a liposome.

11. The topical dermatological formulation of claim 9, wherein said percutaneous chemical permeability enhancer comprises a cyclodextrin.

12. The topical dermatological formulation of claim 9, wherein said percutaneous chemical permeability enhancer comprises a chemically induced heat.

13. The topical dermatological formulation of claim 8, wherein said percutaneous permeability enhancer comprises a physical permeability enhancer.

14. The topical dermatological formulation of claim 13, wherein said percutaneous physical permeability enhancer comprises a dermabrasion.

15. The topical dermatological formulation of claim 13, wherein said percutaneous physical permeability enhancer comprises a microdermabrasion.

16. The topical dermatological formulation of claim 13, wherein said percutaneous physical permeability enhancer comprises an iontophoresis.

17. The topical dermatological formulation of claim 13, wherein said percutaneous physical permeability enhancer comprises a microneedle.

18. The topical dermatological formulation of claim 13, wherein said percutaneous physical permeability enhancer comprises electroporation.

19. The topical dermatological formulation of claim 13, wherein said percutaneous physical permeability enhancer comprises a sonophoresis.

20. The topical dermatological formulation of claim 13 wherein said percutaneous physical permeability enhancer comprises a physically induced heat.
